(19) United States
(12) Patent Application Publication
(10) Pub. No.: US 2025/0195752 A1
(43) Pub. Date: Jun. 19, 2025

(54) FILLABLE MEDICAMENT DELIVERY DEVICE

(71) Applicant: Becton, Dickinson and Company, Franklin Lakes, NJ (US)

(72) Inventors: Nicholas ANDERSON, Westminster, MA (US); Scott STEWART, Somerville, MA (US)

(73) Assignee: Becton, Dickinson and Company, Franklin Lakes, NJ (US)

(21) Appl. No.: 18/545,067

(22) Filed: Dec. 19, 2023

(51) Int. Cl. A61M 5/142; A61M 5/14; A61M 5/145

(52) U.S. Cl. CPC A61M 5/14244; A61M 5/1452; A61M 2005/1403; A61M 2005/14252

(57) ABSTRACT

A fillable medicament delivery device, including a body, a reservoir disposed on the body and having an end wall, and a plunger movably disposed within the reservoir. The device also includes an input port and an output port disposed on one of the end wall and the plunger, and a patient cannula fluidly connected with the output port. The one of the end wall and the plunger has a face oriented toward an interior of the reservoir and the remaining one of the end wall and the plunger, and the face has a channel recessed therefrom. The channel fluidly connects the input port and the output port.

### FIELD OF THE INVENTION

The present invention relates to medical devices, and more particularly, to a fillable medical device with a reservoir and a movable plunger.

### BACKGROUND OF THE INVENTION

The process of filling a reservoir of a medicament delivery device can entrap air in the reservoir. For traditional manual syringes, this issue can be addressed by user training and using gravity to assist by orienting the syringe so that the air rises toward a cannula end of the syringe, thus allowing the user to push in a plunger and expel the air through the cannula.

But the same method is not available for complex micro-dosing syringe pump systems that do not have clear lines of sight to allow detection of possible air entrapment. In related art devices, small variations in mating surfaces between a bottomed out free floating plunger and a reservoir wall in related art devices can be a driving factor for determining fluid flow, likely in the form of splattering around an input port. This uncontrolled behavior is not ideal for guaranteeing air evacuation and preventing air entrapment. Improvement is desirable.

### SUMMARY OF EMBODIMENTS OF THE INVENTION

Accordingly, it is an aspect of the present invention to provide a fillable medicament delivery device with improved ability to expel air entrapped in a reservoir.

The foregoing and/or other aspects of the present invention are achieved by providing a fillable medicament delivery device, including a body, a reservoir disposed on the body and having an end wall, and a plunger movably disposed within the reservoir. The device also includes an input port and an output port disposed on one of the end wall and the plunger, and a patient cannula fluidly connected with the output port. The one of the end wall and the plunger has a face oriented toward an interior of the reservoir and the remaining one of the end wall and the plunger, and the face has a channel recessed therefrom. The channel fluidly connects the input port and the output port.

The foregoing and/or other aspects of the present invention are also achieved by providing a fillable medicament delivery device, including a reservoir, a plunger movably disposed within the reservoir, input and output ports disposed on the plunger, and a patient cannula fluidly connected with the output port.

The foregoing and/or other aspects of the present invention are also achieved by providing a fillable medicament delivery device, including a body, a reservoir disposed on the body and having an end wall with input and output ports disposed therethrough, a plunger movably disposed within the reservoir, and a patient cannula fluidly connected with the output port. The end wall has a channel recessed from an interior surface thereof, the channel fluidly connecting the input port and the output port.

Additional and/or other aspects and advantages of the present invention will be set forth in the description that follows, or will be apparent from the description, or may be learned by practice of the invention.

### BRIEF DESCRIPTION OF THE DRAWINGS

The above and/or other aspects and advantages of embodiments of the invention will be more readily appreciated from the following detailed description, taken in conjunction with the accompanying drawings, of which:

FIG. 1 is a perspective view of a wearable fluid delivery device constructed in accordance with an embodiment of the present invention;

FIG. 2 is a block diagram of example components of a fluid delivery device in accordance with an embodiment of the present invention;

FIG. 3 is a perspective top view of wearable fluid delivery device in accordance with another embodiment of the present invention;

FIG. 4 is a perspective bottom view of the device of FIG. 3;

FIG. 5 is a perspective cross-sectional view of the device of FIG. 3;

FIG. 6 is a cross-sectional view of a reservoir of the device of FIG. 3;

FIG. 7 is a cross-sectional view of the reservoir of FIG. 6 illustrating a channel therein;

FIG. 8 is a partial cross-sectional view of the reservoir of FIG. 3; and

FIG. 9 is a cross-sectional view of the reservoir of FIG. 6 illustrating a channel in a plunger.

### DETAILED DESCRIPTION OF EMBODIMENTS OF THE PRESENT INVENTION

Reference will now be made in detail to embodiments of the present invention, which are illustrated in the accompanying drawings, wherein like reference numerals refer to like elements throughout. The embodiments described herein exemplify, but do not limit, the present invention by referring to the drawings.

The embodiments are not intended to be mutually exclusive so that the features of one embodiment can be combined with other embodiments as long as they do not contradict each other.

It will be understood by one skilled in the art that this disclosure is not limited in its application to the details of construction and the arrangement of components set forth in the following description or illustrated in the drawings. The embodiments herein are capable of other embodiments, and capable of being practiced or carried out in various ways. Phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless limited otherwise, the terms “connected,” “coupled,” and “mounted,” and variations thereof herein are used broadly and encompass direct and indirect connections, couplings, and mountings. In addition, the terms “connected” and “coupled”” and variations thereof are not restricted to physical or mechanical connections or couplings. Further, terms such as “up,” “down,” “bottom,” “top,” “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure.

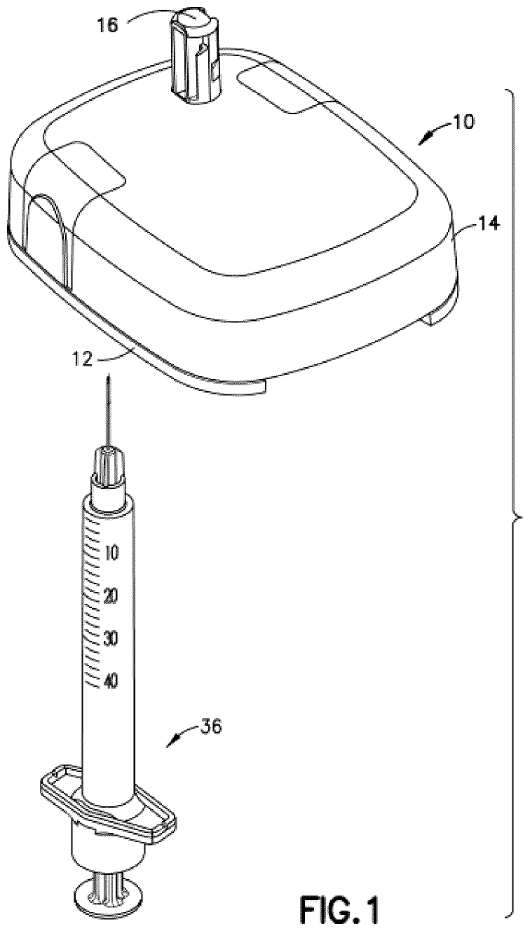

FIG. 1 is a perspective view of one example of a wearable fluid delivery device 10 constructed in accordance with an example embodiment. As shown in FIG. 1, the medicament delivery device 10 comprises a body or baseplate 12, a cover 14, and an insertion mechanism 16 in an un-deployed position. The reservoir fluid delivery device 10 can be filled with the fluid (e.g., medicament or drug) by a user inserting a needle of a filled syringe 36 into a fill port (not shown) provided in the baseplate 12 that has an inlet fluid path from the fill port to the reservoir. It is to be understood that the fluid delivery device 10 can be filled with a fluid (e.g., drug) using different mechanisms and methods.

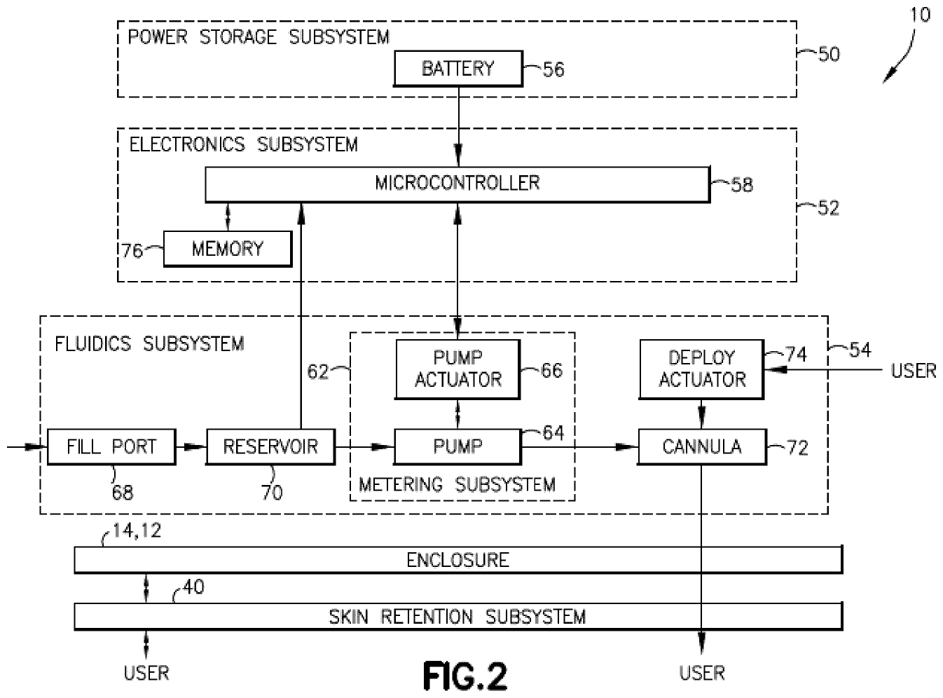

FIG. 2 is an illustrative system diagram that illustrates example components in an example medication delivery device 10 having for example, an infusion pump. The medication delivery device 10 can include an electronics sub-system 52 for controlling operations of components in a fluidics sub-system 54 such as the pump 64. A power storage sub-system 50 can include one or more batteries 56, for example, for providing power to components in the electronics and fluidics sub-systems 52 and 54. The fluidics sub-system 54 can comprise, for example, an optional fill port 68 for filling a reservoir 70 (e.g., with medication), although the medication delivery device 10 can be optionally shipped from a manufacture having its reservoir already filled, or can be configured to receive a filled reservoir from a medical professional or user. The medication delivery device 10 also includes an insertion mechanism 74 for deploying a cannula 72 for insertion into an infusion site on a patient's skin. The fluidics sub-system 54 also has a metering sub-system 62 comprising the pump 64 and a pump actuator 66.

The pump actuator 66 can be a DC motor and gearbox assembly or other pump driving mechanism for controlling the plunger or piston 30. The microcontroller 58 can be provided with an integrated or separate memory device 76 having computer software instructions for controlling, for example, operation of the pump actuator 66.

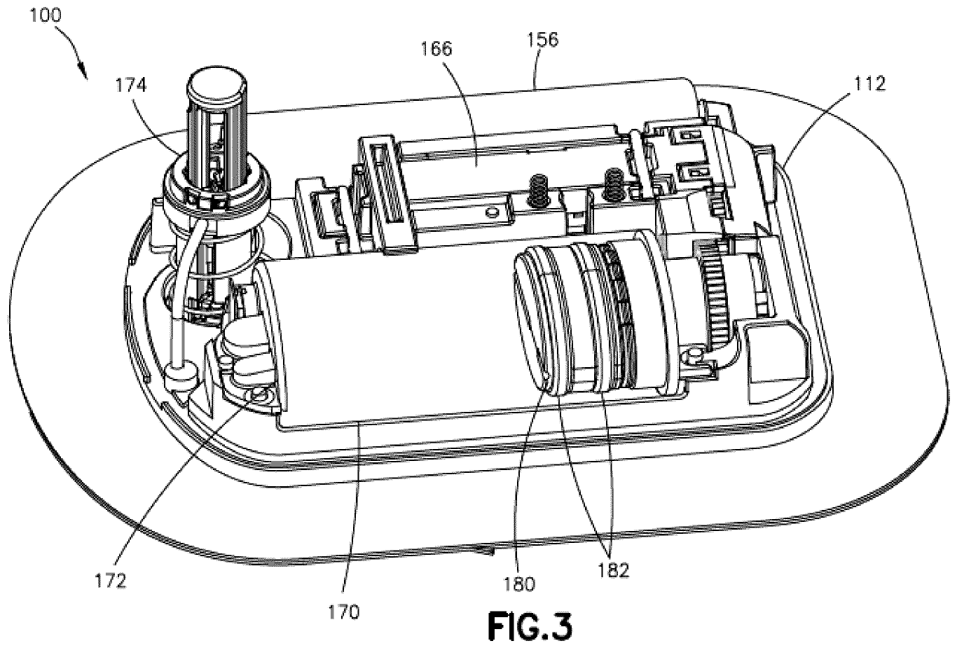

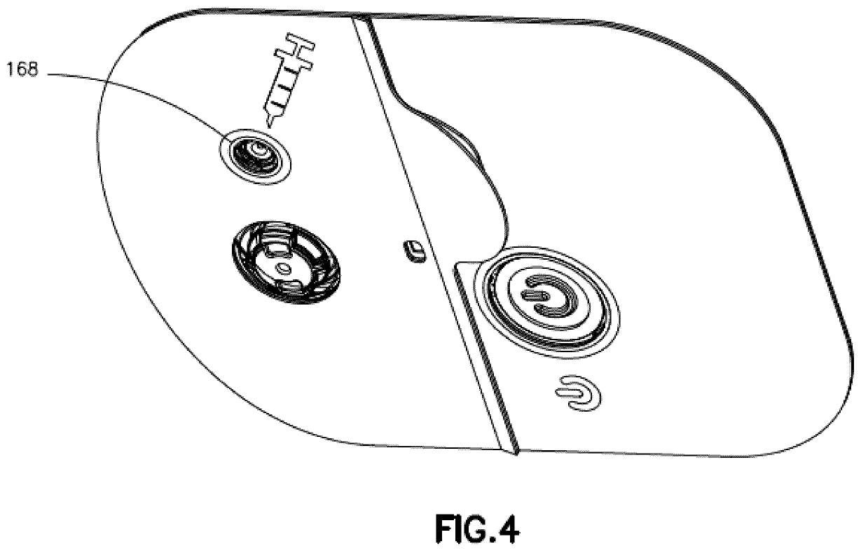

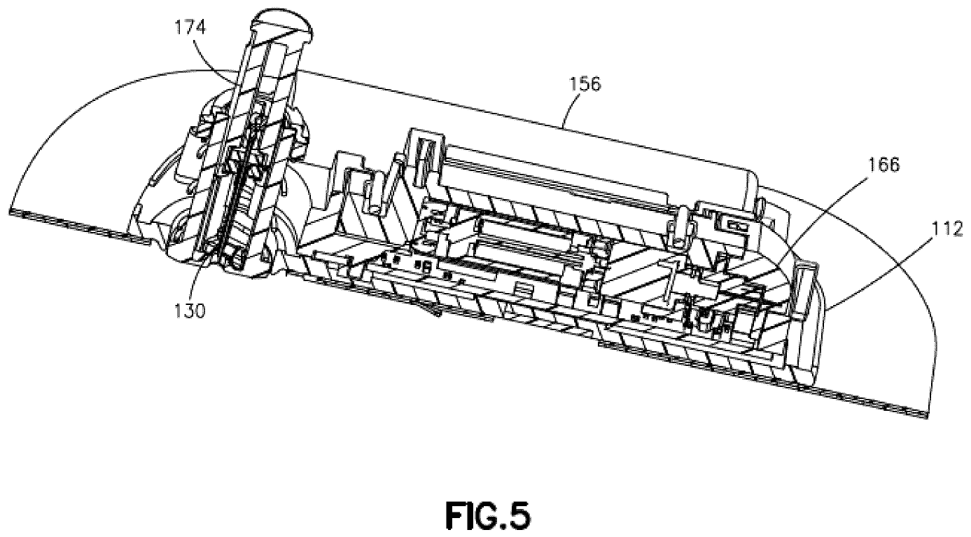

FIG. 3 is a perspective view of wearable fluid delivery device 100 in accordance with another embodiment of the present invention, in which a cover 114 is removed to aid clarity. Similar to the embodiment of FIGS. 1 and 2, the device 100 includes a baseplate or body 112 on which one or more batteries 156, a drive actuator 166 (such as a DC motor and gearbox assembly or other driving mechanism), an insertion mechanism 174, and a reservoir 170 are mounted. According to one embodiment, the reservoir 170 is fixedly mounted to the body 112. The device 100 also includes a patient cannula 130 (best shown in FIG. 5), as part of the insertion mechanism 174.

Preferably, the reservoir 170 is rigid. The reservoir 170 may be unitarily formed from non-metallic and metallic materials, such as polymeric materials, including, but not limited to, thermoplastics, stainless steels or other metallic alloys.

The reservoir 170 has a stopper or plunger 180 movably disposed therein. According to one embodiment, the stopper is freely movable within the reservoir 170. The stopper 180 is not connected to a driving mechanism that would inhibit the motion of the stopper 180, and the stopper 180 moves within the reservoir as a result of the pressure of the fluid (e.g., medicament) in the reservoir. According to one embodiment, prior to filling, the stopper is freely movable within the reservoir 170. Subsequent to filling with medicament, a driving mechanism, such as drive actuator 166, engages the stopper to drive the stopper to dispense the medicament.

For example, according to one embodiment, the reservoir 170 is initially filled by a patient or medical professional using a syringe 36 to create volumetric displacement of the delivery fluid. This is achieved by starting the stopper 180 at a distal or front end of the reservoir 170 with a completely empty reservoir 170. The stopper 180 is pushed back by entering fluid that is introduced by the patient via the fill port 168. That is, when the reservoir 170 is filled by the syringe 36 via the fill port 168, the stopper 180 is driven toward a proximal or rear of the reservoir 170 by the incoming fluid.

The stopper 180 is a sealing member that may be made up of one or several components to create a floating piston face. The plunger 180 may have more than one contact point for sealing along a longitudinal axis of the reservoir 170 to balance forces and prevent tilting of the plunger 180 relative to the reservoir 170. The plunger 180 may be unitarily formed from non-metallic and metallic materials, such as polymeric materials, including, but not limited to, thermoplastics, stainless steels or other metallic alloys. The plunger 180 may also have O-ring seals 182 present to achieve a robust sealing joint with the reservoir 170.

According to one embodiment, the reservoir 170 has an end wall 172 with an input port 176 and an output port 178 disposed threrethrough. The end wall has a face 173 oriented toward an interior of the reservoir 170 and the plunger 180, and the face 173 has a channel 190 recessed therefrom. The channel 190 fluidly connects the input port 176 and the output port 178.

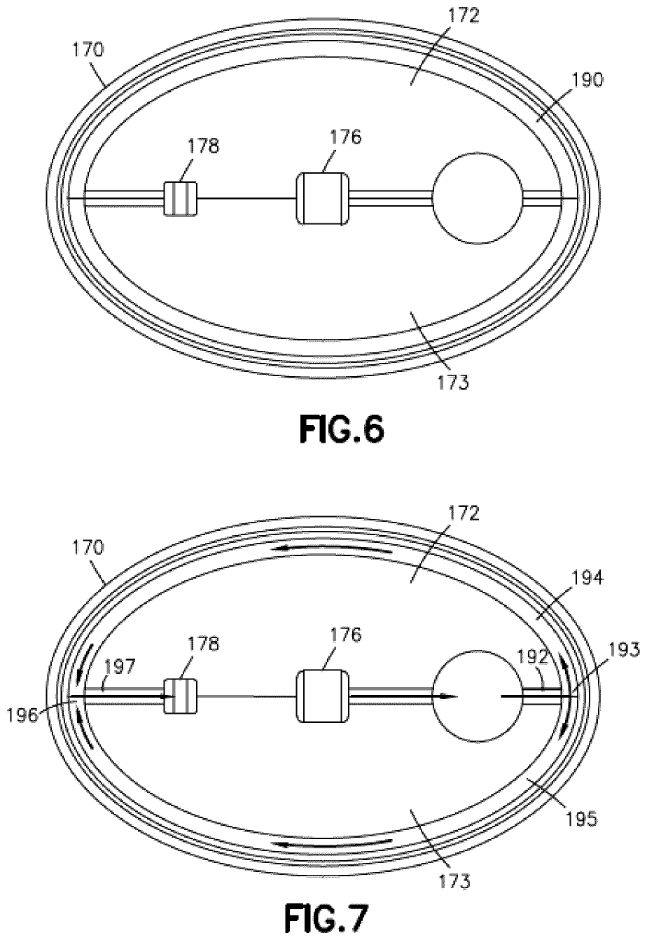

More specifically, as shown in FIG. 7, the channel 190 includes a first lateral portion 192 from the input port 176 toward a first intersection 193 at an interior wall of the reservoir 170. From the first intersection 193, the channel 190 splits in opposite directions into first and second perimeter portions 194 and 195 that travel around a perimeter of the face 173 to a second intersection 196. The first and second perimeter portions 194 and 195, rejoin at the second intersection 196. From the second intersection 196, the channel 190 includes a second lateral portion 197 to the output port 178. The output port 178 is fluidly connected with the patient cannula 130.

According to one embodiment, there is a downstream flowpath from the output port 178 through the patient cannula 130, and a selectively permeable membrane is disposed in the downstream flowpath.

In operation, prior to filling the reservoir 170, the plunger 180 is positioned immediately adjacent to the face 173. According to one embodiment, the plunger 180 is positioned to contact the face 173 of the end wall 172. During filling of the reservoir 170, the shape and position of the channel 190 directs the fluid (medicament) entering the reservoir 170 through the input port 176 to the output port 178 to drive air out of the reservoir 170 prior to moving the plunger 180 and at least partially filling a remainder of the reservoir 170.

According to one embodiment, a pressure required to push the medicament through the selectively permeable membrane is greater than a pressure required to move the plunger 180. Thus, when medicament is introduced through the input port 176, once the air is driven out of the reservoir 170 by the medicament flow through the channel 190, and driven through the selectively permeable membrane, the fluid pressure only builds in the downstream flowpath and the reservoir 170 once the fluid (medicament) reaches the selectively permeable membrane. At this point, because the pressure required to move the plunger 180 is less than the pressure required to get the fluid past the selectively permeable membrane, the plunger 180 is moved in the reservoir by the fluid entering the reservoir 170 via the input port 176. Once the plunger 180 bottoms out at the back of the reservoir 170, continued fluid entry via the input port 176 causes the pressure in the reservoir and downstream flowpath to increase to a point that the fluid can pass through the selectively permeable membrane, thereby demonstrating that the device 100 is primed. Subsequently, operation of the drive actuator 166 drives the plunger forward to dispense the fluid.

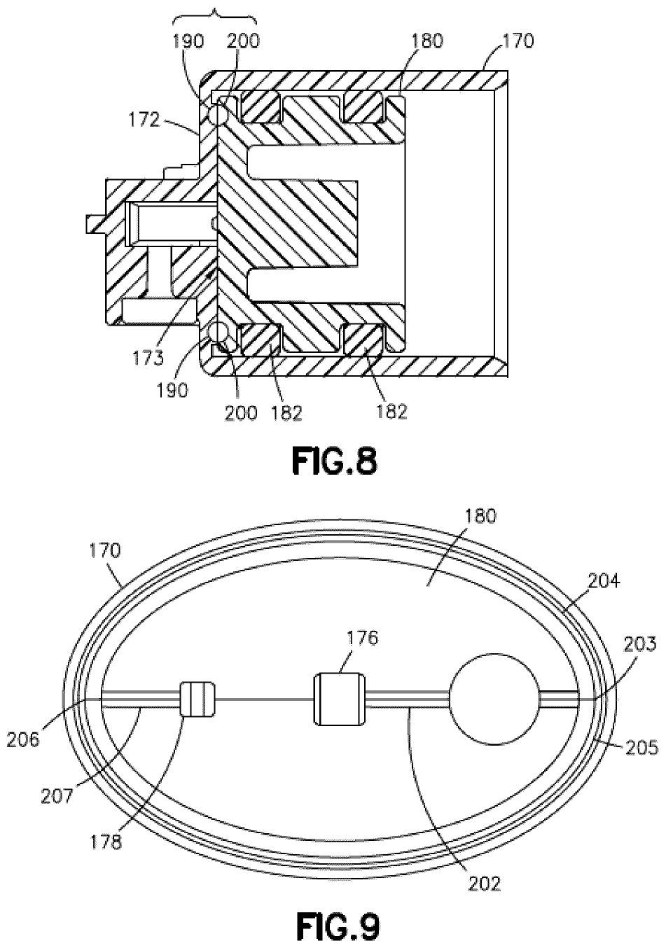

Preferably, a cross-sectional shape of the channel 190 is semicircular, as best shown in FIG. 8. One skilled in the art will appreciate, however, that the channel 190 can have a different cross-sectional shape without departing from the present invention's scope.

According to another embodiment, as shown in FIG. 9, instead of the end wall 170 having the input and output ports 176 and 178, it is the plunger 180 that has the input and output ports 176 and 178 disposed thereon (therethrough).

According to another embodiment, the plunger 180 also has a plunger face 183 oriented toward the interior of the reservoir 170 and the face 173 of the end wall 172. The plunger face 183 has a plunger channel 200 recessed therefrom. Similar to the previous embodiment, the plunger channel 200 includes a first lateral portion 202 from the input port 176 toward a first intersection 203 at an interior wall of the reservoir 170. From the first intersection 203, the plunger channel 200 splits in opposite directions into first and second perimeter portions 204 and 205 that travel around a perimeter of the face 183 to a second intersection 206. The first and second perimeter portions 204 and 205, rejoin at the second intersection 206. From the second intersection 206, the plunger channel 200 includes a second lateral portion 207 to the output port 178. The output port 178 is fluidly connected with the patient cannula 130.

According to one embodiment, there is a downstream flowpath from the output port 178 through the patient cannula 130, and a selectively permeable membrane is disposed in the downstream flowpath.

In operation, prior to filling the reservoir 170, the plunger face 183 of the plunger 180 is positioned immediately adjacent to the face 173. According to one embodiment, the plunger face 183 is positioned to contact the face 173 of the end wall 172. During filling of the reservoir 170, the shape and position of the plunger channel 200 directs the fluid (medicament) entering the reservoir 170 through the input port 176 to the output port 178 to drive air out of the reservoir 170 prior to moving the plunger 180 and at least partially filling a remainder of the reservoir 170.

According to one embodiment, the pressure required to push the medicament through the selectively permeable membrane is greater than a pressure required to move the plunger 180. Thus, when medicament is introduced through the input port 176, once the air is driven out of the reservoir 170 by the medicament flow through the plunger channel 200, and driven through the selectively permeable membrane, the fluid pressure only builds in the downstream flowpath and the reservoir 170 once the fluid (medicament) reaches the selectively permeable membrane. At this point, because the pressure required to move the plunger 180 is less than the pressure required to get the fluid past the selectively permeable membrane, the plunger 180 is moved in the reservoir by the fluid entering the reservoir 170 via the input port 176. Once the plunger 180 bottoms out at the back of the reservoir 170, continued fluid entry via the input port 176 causes the pressure in the reservoir and downstream flowpath to increase to a point that the fluid can pass through the selectively permeable membrane, thereby demonstrating that the device 100 is primed. Subsequently, operation of the drive actuator 166 drives the plunger forward to dispense the fluid.

Preferably, a cross-sectional shape of the plunger channel 200 is semicircular, as best shown in FIG. 8. One skilled in the art will appreciate, however, that the plunger channel 200 can have a different cross-sectional shape without departing from the present invention's scope.

According to yet another embodiment, the input and output ports 176 and 178 can be disposed in one of the end wall 172 and the plunger 180, but both the end wall 172 and the plunger 180 respectively include the channel 190 and the plunger channel 200, as shown in FIG. 8. In this embodiment, prior to filling the reservoir, the interior surface of the end wall 172 (face 173) and the plunger face are disposed adjacent to each other, and the channel 190 and the plunger channel together form a combined channel 210.

Embodiments of the present invention employ flow channel features of appropriate size and shape to connect the input port 176 and output port 178 in specific coordination to control flow direction such that fluid wicks uniformly throughout the reservoir 170 and plunger 180 design combination, pushing a majority of the air in the system toward the output port 178 during a fill operation. To achieve the high accuracy of micro-dosing syringe pumps, these design features are intended to prevent air from remaining trapped in the reservoir 170 itself. When air is properly evacuated to the output port before fluid reaches the same location, all of that air can be permanently evacuated from the overall system via one or more selectively permeable membranes that are integrated into the downstream flowpath past the output port 178.

Embodiments of the present invention can include channel features that are part of the free floating plunger 180, the reservoir end wall 170, or both. Either interface (or the combination) will create a flow channel that reduces the probability of random splatter of fluid due to very small component gaps by providing a fluidic path of least resistance. Embodiments of the present invention aim to reduce the occurrence rate of air introduction due to system architecture and geometry.

Embodiments of the present invention may be adapted to optimize the size, quantity and shape of the flow channels to allow for timely and steady flow to be achieved between the input and output ports given a range of input pressures. Position of the input and output ports may change the orientation of the features. Channels of these embodiments are ideally located in areas where stagnant air entrapment regions are likely to occur, such as the plunger and seal gap that may be present. Ideally, the channels have a continuous path that has a clear start at the input port and ends at the output port. Embodiments of the present invention also benefit from symmetry in channel design to ensure that if there are splits in the path, both paths arrive at the output port at the same time.

Although only a few embodiments of the present invention have been shown and described, the present invention is not limited to the described embodiments. It will be appreciated by those skilled in the art that other changes may also be made to the disclosed embodiments without departing from the scope of the invention. In addition, any of the embodiments, features and/or elements disclosed herein may be combined with one another to form various additional combinations not specifically disclosed, as long as the embodiments, features and/or elements being combined do not contradict each other. All such changes and combinations are considered to be within the scope of the invention as defined by the appended claims and their equivalents.

Various aspects of the multiple embodiments may be employed independently or in combinations thereof.

What is claimed is:

1. A fillable medicament delivery device, comprising:
a body;
a reservoir disposed on the body and having an end wall;
a plunger movably disposed within the reservoir;
an input port disposed on one of the end wall and the plunger;
an output port disposed on the one of the end wall and the plunger; and
a patient cannula fluidly connected with the output port,
wherein the one of the end wall and the plunger has a face oriented toward an interior of the reservoir and the remaining one of the end wall and the plunger, and the face has a channel recessed therefrom, the channel fluidly connecting the input port and the output port.

2. The device according to claim 1, wherein the channel comprises:
a first lateral portion from the input port toward a first intersection at an interior wall of the reservoir;
first and second perimeter portions travelling in opposite directions from the first intersection around a perimeter of the face to a second intersection; and
a second lateral portion from the second intersection to the output port.

3. The device according to claim 1, wherein a shape and a position of the channel directs fluid entering the reservoir through the input port to the output port prior to at least partially filling a remainder of the reservoir to drive air out of the reservoir.

4. The device according to claim 1, wherein the plunger is freely movable within the reservoir.

5. The device according to claim 4, wherein a shape and a position of the channel directs fluid entering the reservoir to the output port to drive air out of the reservoir prior to moving the plunger and at least partially filling a remainder of the reservoir.

6. The device according to claim 1, wherein a cross-sectional shape of the channel is semicircular.

7. The device according to claim 1, wherein the remaining one of the end wall and the plunger has a secondary face oriented toward the interior of the reservoir and the one of the end wall and the plunger, and the secondary face has a secondary channel recessed therefrom.

8. The device according to claim 7, wherein the secondary channel has a shape corresponding to the channel.

9. The device according to claim 7, wherein cross-sectional shapes of the channel and the secondary channel are each semicircular.

10. The device according to claim 7, wherein prior to filling the reservoir, the face and the secondary face are disposed adjacent to each other, and the channel and the secondary channel together form a combined channel.

11. The device according to claim 10, wherein a cross-sectional shape of the combined channel is circular.

12. The device according to claim 1, wherein the a body has a fill port disposed thereon; and
the input port is fluidly connected with the fill port.

13. A fillable medicament delivery device, comprising:
a reservoir;
a plunger movably disposed within the reservoir;
an input port disposed on the plunger;
an output port disposed on the plunger; and
a patient cannula fluidly connected with the output port.

14. The device according to claim 13, wherein the plunger has a face oriented toward an interior of the reservoir, the face having a channel recessed therefrom and fluidly connecting the input and output ports.

15. The device according to claim 13, wherein the plunger is freely movable within the reservoir.

16. The device according to claim 14, wherein the channel comprises:
a first lateral portion from the input port to a first intersection at an interior wall of the reservoir;
first and second perimeter portions travelling in opposite directions from the first intersection around a perimeter of the face to a second intersection; and
a second lateral portion from the second intersection to the output port.

17. A fillable medicament delivery device, comprising:
a body;
a reservoir disposed on the body and having an end wall with input and output ports disposed therethrough;
a plunger movably disposed within the reservoir; and
a patient cannula fluidly connected with the output port,
wherein the end wall has a channel recessed from an interior surface thereof, the channel fluidly connecting the input port and the output port.

18. The device according to claim 17, wherein the channel comprises:
a first lateral portion from the input port toward a first intersection at an interior wall of the reservoir;
first and second perimeter portions travelling in opposite directions from the first intersection around a perimeter of the face to a second intersection; and
a second lateral portion from the second intersection to the output port.

19. The device according to claim 17, wherein the plunger has a plunger face oriented toward the interior surface of the end wall, the plunger face having a plunger channel recessed therefrom, the plunger channel having a shape corresponding to the channel.

20. The device according to claim 19, wherein prior to filling the reservoir, the interior surface of the end wall face and the plunger face are disposed adjacent to each other, and the channel and the plunger channel together form a combined channel.
